(19) United States
(12) Patent Application Publication
(10) Pub. No.: US 2019/0015605 A1
(43) Pub. Date: Jan. 17, 2019

(54) Phone Puff - Vaporizer with Data Collection

(71) Applicant: Daniel Palmer, Mentone, CA (US)

(72) Inventor: Daniel Palmer, Mentone, CA (US)

(21) Appl. No.: 16/036,903

(22) Filed: Jul. 16, 2018

Related U.S. Application Data
(63) Continuation of application No. 61/984,020, filed on Apr. 24, 2014, now abandoned.
(60) Provisional application No. 62/538,765, filed on Jul. 30, 2017.

(51) Int. Cl. A61M 11/04; G06F 21/32

(52) U.S. Cl. CPC A61M 11/042; G06F 21/32; A61M 2205/505; A61M 2205/3592; A61M 2205/50; A61M 2205/8206

(57) ABSTRACT

A cartridge which contains medicine/substance that is consumed by heating and inhaling when connected to a smartphone charging port for heating power and application control of user access, dosing, and data collection. The duration of use is limited to smartphone charging port connectivity, eliminating risk of accidental overheating. Application control of access ensures user and dosing specifications, preventing non-authorized user/accidental excess consumption. Data collection via this method of consumption informs and guides subsequent user cartridge/dosing decisions and recommendations.

### BACKGROUND OF INVENTION

Modern vaporizer devices entail multiple components (such as batteries and chargers) or a combined apparatus with risk of overheating. These devices lack integrated data collection functionality with regard to dose, type, and user. In addition, security of substance is variable among devices, with incorporation of no, or overly simple, “child safe” features.

### SUMMARY OF INVENTION

This invention consolidates vaporizer components while eliminating risk of overheating. It integrates data collection regarding dose, type, and user with secure access to consumption via inhalation when connected to a smartphone charging port.

### BRIEF DESCRIPTION OF FIGURES

FIG. 1 is a front view of this invention.

### DETAILED DESCRIPTION OF THE INVENTION

Phone Puff Cartridges

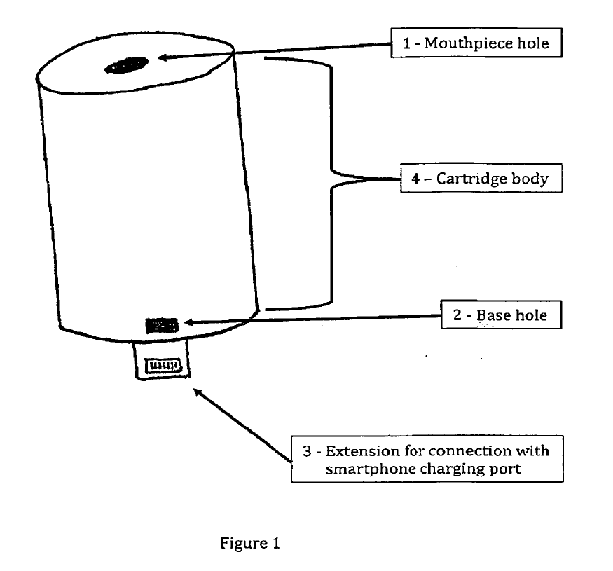

These will contain medicine/substance that is used by heating and inhaling (FIG. 1, #1 and #2). Expect a large market for cannabinoid-containing substances, but also others including antibiotics (pneumonia-related, for example). Inhalable versions can replace pill forms of medication, with multiple benefits including reduced toxicity and faster effects. Cartridges with cannabinoid-containing substances can replace products with unwanted side effects, including medications as well as recreational drugs such as alcohol, smoked tobacco, and even smoked marijuana. One anticipated outcome of widespread availability of the Phone Puff is a decrease in the frequency of fetal alcohol syndrome.

Standard Use of Cartridge/App:

Plug cartridge into phone charging port (FIG. 1, #3). Phone/app should recognize cartridge. Possibly type in cartridge/product code if needed. App will bring up 3 choices as to why you are about to use product, small option to “skip” at bottom. Question may be: which of the following best matches your reason for this current usage? Options may include, for example, feeling “nervous” or “panicked” or “depressed” etc. (depends on user profile and cartridge info). For some choices, app may recommend that you switch to a different cartridge. It could say, “***** cartridge may be more helpful. Feel free to switch if desired.” Along with this text will be a large button/option to “continue.” if “continue” is pressed, everything will proceed as if there had not been that recommendation. Power will flow from phone battery into substance heater, OR into a power reserve that will be RECHARGING the battery that was filled up right after last use. A minute or few of charging will give several seconds of heating power, ideally FOR NEXT TIME. Probable small battery or capacitor in cartridge with energy for one use. The cartridge will be ready to go immediately if it had been charged last time (assuming multiple uses per cartridge). After use, user may need to leave it plugged in to charge for a few minutes. Cartridge body (FIG. 1, #4) is between one and two inches in length and may have a physical button that can be pushed to cause the actual heating (or a button on the touch screen once technologically achieved). We want the cartridge to only work when plugged into the phone, so we can get as much data as possible about dosing and effectiveness, and prevent children or non-authorized persons from using cartridge.

Some options will appear on the phone a bit before the charging (if necessary) is complete and/or at a specified time increment (15 minutes, for example) after consumption. The screen will say, “How well has this dose helped your symptoms?” with options of “not helped,” “helped a little,” “helped a lot,” and an option at the top (with a check mark box next to it) that says “Check this box first if dose had benefits unrelated to symptoms.” After this, it could say, “Thank you! Your participation helps improve medical knowledge.” And perhaps, “Your cartridge is now charged.”

More about software: The three options of symptoms will be placed in random order each time, so that if a subset of users is simply clicking the middle button as fast as possible, or something like that, to get to the dose quicker, those random answers will be distributed roughly equally among the choices. Phone Puff can be used when the phone is out of service; information regarding previous usage will be sent to us next time the phone connects.

When First Installing App:

Welcome screen includes: Thank you for installing Phone Puff . . . we appreciate your being a responsible user of these (doses/medications/products) and all feedback we get from you will be used to improve medical knowledge. The following (questions/options) are anonymous and optional and will not affect your ability to use any (cartridge/product). We appreciate your answering as much as you can. Separate screens: Age, Gender, Height, Race, Weight. We will choose these options wisely and perhaps let the user choose more than one per screen.

What cartridges/strains are recommended for what symptoms/conditions/ailments can be adjusted based on feedback. We will keep track of which situations cause questions to be “skipped” more often and adjust our strategies and/or options and/or questions based on that information.

Perhaps user will need to click on Phone Puff app before (or after) plugging in cartridge, rather than cartridge opening app automatically. We don't want it to seem like something that takes over the phone. If user gets phone call to device while app is open (probably) let phone call appear, goes back to app immediately after phone call ends or is rejected or ringing stops (the norm).

RECALLS: We will update users' phones so that if a certain group of cartridges is recalled for any reason, and one of those cartridges is plugged in, the user will see a message about the recall, and be informed that the current cartridge can be exchanged for a replacement.

1. Battery power for heating/vaporization of substance for consumption of inhaled doses comes from smartphone battery, eliminating the need for multiple separate components to achieve a functional vaporization apparatus (such as batteries and chargers). This consolidated functionality is an improvement to contemporary vaporization approaches.

2. Data collection of vaporized inhaled dose amount, frequency, type, user demographics, and consumption rationale through this consolidated vaporization apparatus in conjunction with smartphone technological capabilities is an improvement to contemporary data collection approaches and capabilities, enabling feasible individual data synthesis and large-scale pooled data synthesis and double-blind trials.

3. Security and safety of substance via containment in and access from this device is an improvement to contemporary vaporization approaches, as device preserves substance from contamination, minimizes tamperability, quantifies dosing, and allows access to consumption only to individual(s) entering a passcode, an authorized fingerprint, etc. on the smartphone vaporizer application.
